(19) United States
(12) Plant Patent Application Publication
(10) Pub. No.: US 2011/0185460 P1
(43) Pub. Date: Jul. 28, 2011

(54) Blueberry plant named 'Ridley 1104'

(50) ;

(76) Inventor: Ridley Bell, Lindendale (AU)

(21) Appl. No.: 12/930,908

(22) Filed: Jan. 18, 2011

Related U.S. Application Data
(60) Provisional application No. 61/336,458, filed on Jan. 22, 2010.

(51) Int. Cl. A01H 5/00

(52) U.S. Cl. PLT/157

(57) ABSTRACT

A new and distinct cultivar of Vaccinium hybrid plant named ‘RIDLEY 1104’, characterized by its combination of spreading plant shape and strong plant growth vigor, evergreen foliage with broad leaves, early to medium timing of flowering and fruiting, medium to large fruit size, firm fruit suited to handling, attractive dark blue fruit color, medium to high berry sweetness and small picking scar.

### LATIN NAME OF GENUS AND SPECIES OF PLANT CLAIMED

Vaccinium hybrid

### VARIETY DENOMINATION

‘Ridley 1104’

### BACKGROUND OF THE INVENTION

The present invention relates to a new and distinct perennial variety of Vaccinium hybrid (Southern Highbush Blueberry), which has been given the variety denomination of ‘Ridley 1104’. Its market class is that of a fruiting plant. ‘Ridley 1104’ is intended for use as fresh fruit for shipping, customer pick and processing markets and as a home garden plant.

The new Vaccinium hybrid cultivar is a selection resulting from seedlings produced in a controlled breeding programme of Vaccinium at Lindendale, NSW, Australia in 2003 from the controlled pollination of seed parent “C97-390” (U.S. Plant Pat. No. 20,642) and pollen parent “C97-41” (patent pending). The new cultivar was selected as a single plant within a population of plants resulting from this controlled pollination of Vaccinium hybrids in 2005 in a commercial field plantation environment at Lindendale, New South Wales, Australia. Selection criteria was a combination of strong plant growth vigor, low chilling requirement, early to medium season of flowering and fruiting, firm fruit suited to handling, sweet berries and small picking scar. The selection was subsequently evaluated for a number of years at the commercial farms of Mountain Blue Orchards at Lindendale, New South Wales, Australia.

Asexual reproduction of the new cultivar by cutting propagation since 2005 at Lindendale, New South Wales, Australia and has demonstrated that the new cultivar reproduces true to type with all of the characteristics, as herein described, firmly fixed and retained through successive generations of such asexual propagation.

The new variety can be compared to the seed parent “C97-390” (U.S. Plant Pat. No. 20,642). In Lindendale, New South Wales, Australia the time of both flowering and fruit ripening of ‘Ridley 1104’ is classified as ‘early to medium’ whereas the time of flowering and fruit ripening of ‘C97-390’ is classified as ‘very early to early’. The berry diameter of ‘Ridley 1104’ is ‘medium to large whereas the berry diameter of ‘C97-390’ is ‘medium’. The new variety can also be compared to the pollen parent “C97-41” (U.S. patent application pending). In Lindendale, New South Wales, Australia the time of flowering of ‘Ridley 1104’ is classified as ‘early to medium’ whereas the time of flowering of ‘C97-41’ is classified as ‘early’. The berry diameter of ‘Ridley 1104’ is ‘medium to large whereas the berry diameter of ‘C97-41’ is ‘medium’.

The new variety has been primarily compared to the variety ‘C99-42’ (U.S. Plant Pat. No. 20,695) In Lindendale, New South Wales, Australia the time of beginning of flowering of ‘Ridley 1104’ is classified as ‘early to medium’ whereas the time of beginning of flowering of time of ‘C99-42’ is classified as ‘very early to early’. The time of fruit ripening of ‘Ridley 1104’ is classified as ‘early to medium’ whereas the time of fruit ripening of ‘C99-42’ is classified as ‘early’. The leaf length of ‘Ridley 1104’ is ‘medium to long’ whereas the leaf length of ‘C99-42’ is ‘medium’. The leaf width of ‘Ridley 1104’ is ‘broad’ whereas the leaf width of ‘C99-42’ is ‘narrow to medium’. The berry diameter of ‘Ridley 1104’ is ‘medium to large (mean 16.2 mm) whereas the berry diameter of ‘C99-42’ is ‘medium’ (mean 15.5 mm). The berry sweetness of ‘Ridley 1104’ is ‘medium to high’ whereas ‘C99-42’ is ‘low to medium’. This combination of vegetative and fruiting attributes results in ‘Ridley 1104’ being a desirable variety for very early to medium season commercial production. See Table 1 for comparisons.

‘Ridley 1104’ can be compared to the variety ‘Star’ (U.S. Plant Pat. No. 10,675). The plant growth vigour of ‘Ridley 1104’ is ‘strong’ whereas the plant growth vigour of ‘Star’ is ‘medium’. The plant growth habit of ‘Ridley 1104’ is spreading whereas the plant growth habit of ‘Star’ is upright. The leaf length of ‘Ridley 1104’ is medium to long whereas the leaf length of ‘Star’ is medium. The leaf width of ‘Ridley 1104’ is broad whereas the leaf length of ‘Star’ is narrow. The berry diameter of ‘Ridley 1104’ is medium to large (mean 16.2 mm) whereas the berry diameter of ‘Star’ is large (mean 18.1 mm). The average berry weight of ‘Ridley 1104’ is 1.9 g whereas the average berry weight of ‘Star’ is 2.3 g. The berry calyx basin diameter of ‘Ridley 1104’ is small to medium (mean 5.5 mm) whereas the berry calyx basin diameter of ‘Star’ is large to very large (mean 8.8 mm). The berry calyx basin depth of ‘Ridley 1104’ is deep whereas the berry calyx basin depth of ‘Star’ is shallow. The berry sweetness of ‘Ridley 1104’ is ‘medium to high’ whereas ‘Star’ is ‘low to medium’.

### SUMMARY OF THE INVENTION

The following characteristics of the new cultivar have been repeatedly observed and can be used to distinguish ‘RIDLEY 1104’ as a new and distinct cultivar of Vaccinium hybrid plant:
1. Spreading plant growth habit
2. early to medium timing of beginning of flowering
3. early to medium timing of fruit ripening
4. Strong plant growth vigor
5. Dark blue berry color
6. Berry firmness is firm
7. Picking scar is dry
8. Leaf width is broad
9. Berry size is medium to large
10. Berry sweetness is medium to high

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs differ slightly from the color values cited in the detailed description, which accurately describes the colors of ‘RIDLEY 1104’.

FIG. 1 illustrates a ‘RIDLEY 1104’ bush in a commercial field planting.

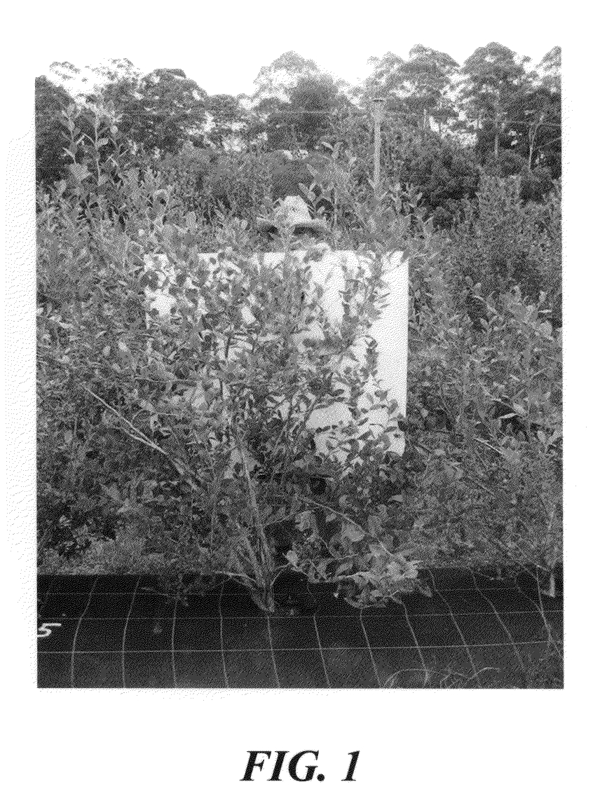

FIG. 2 illustrates a ‘RIDLEY 1104’ leaf, berry and flower shape and dimensions.

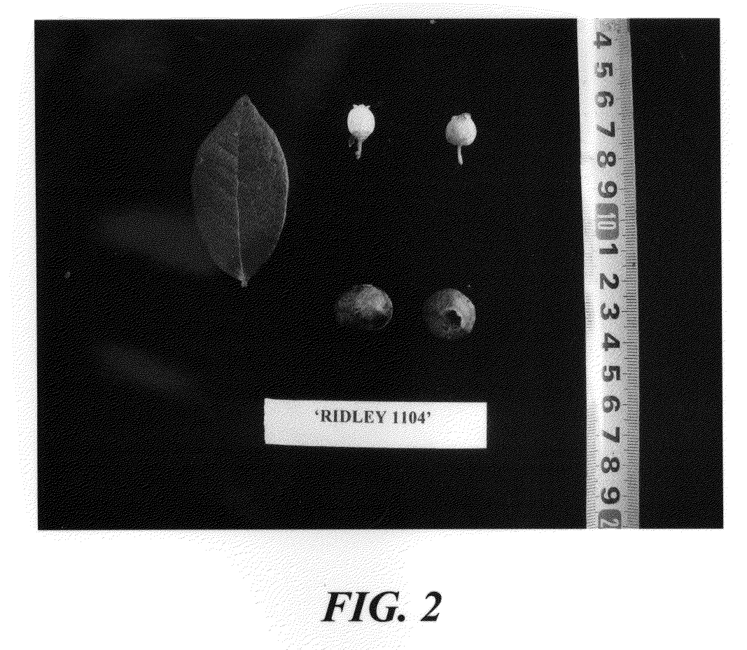

### DETAILED BOTANICAL DESCRIPTION

The following is a detailed botanical description of a new and distinct variety of a Vaccinium hybrid ornamental plant known as ‘RIDLEY 1104’. Plant observations were made on plants grown in Lindendale, New South Wales, Australia. Unless indicated otherwise, the descriptions disclosed herein are based upon observations made in August 2009 of mature ‘RIDLEY 1104’ plants (age 4 years) grown in outdoor field plantings with day temperature ranging from 21° C. to 23° C., night temperatures ranging from 10° C. to 12° C., and light levels ranging from 6 to 8 klux. Those skilled in the art will appreciate that certain characteristics will vary with older or, conversely, younger plants. ‘RIDLEY 1104’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light quality, light intensity, day length, cultural conditions and the like. Color notations are based on The Royal Horticultural Society Color Chart, of The Royal Horticultural Society, London, 2007 edition.
Botanical classification: Vaccinium hybrid cultivar RIDLEY 1104.
Parentage:
   Parents.—seed parent “C97-390” (U.S. Plant Pat. No. 20,642); pollen parent “C97-41” (U.S. patent pending).
Plant:
   Growth habit.—spreading. Growth vigor is strong. Plant height is approximately 1.9 m. Plant width is approximately 1.5 m. Flower bud density is medium. Twigginess is low. Time of beginning of flowering is early to medium (begins early May; peaks late July) when grown as an evergreen in Australia. Time of fruit ripening is early to medium (begins mid June; peaks late September) when grown as an evergreen in Australia.
Stem:
   Suckering tendency.—Plants typically have 5-7 major canes per plant from a base 25 cm in diameter on 5 year old plants.
   Canes.—semi-erect with moderate branching; color greyed orange 198C; 2 year old cane diameter 12 mm, colored greyed oiange 177B, some flaking of bark, current season wood diameter 3-4 mm, colored yellow green 144C Internode length on strong, new shoots 15-20 mm.
   Fruiting wood.—numerous twigs to 10 cm in length.
   Surface texture of new wood.—smooth.
Fully developed leaf: Length medium to long 60-70 mm Width broad approximately 40 mm Shape elliptic Color yellow green 137A Intensity of color medium Margin entire Undulation of margin weak Pubescence of upper side absent Pubescence of lower side absent Cross sectional profile flat Longitudinal profile straight Attitude broad acute to horizontal.
Inflorescence: Length of peduncle to 12 mm Length of pedicel long 4-6 mm Number of flowers per cluster median 7 Flower cluster density is medium.
Flowers: Length of corolla tube medium to long (to 15 mm) Width of corolla tube medium to broad (to 11 mm) Corolla aperture diameter 3-4 mm Color of corolla white 155D Anthocyanin coloration of corolla absent Corolla ridges present Protusion of stigma absent Fragrance weak Shape urceolate Abundance of pollen high Calyx (with sepals) diameter 5-6 mm Calyx color green 138A.
   Productivity.—3 to 4 kg per season from 3-4 year old plants planted at 3.0 m×0.9 m density.
   Cold hardiness.—RIDLEY 1104 has not been grown in all environments including harsh winter environments. Cold tolerance is expected to be low.
   Chilling requirement.—RIDLEY 1104 has not been grown in all environments and is typically grown as an evergreen crop where chilling hours are not important. RIDLEY 1104 is classed as ‘low chill’, typical of Southern Highbush Blueberry varieties with an estimated chilling requirement of 250 hours (not tested).
   Leafing.—plants retain leaves year round (at Lindendale, NSW Australia) and RIDLEY 1104 is grown commercially as an evergreen crop. New leafing occurs in mid to late August at Lindendale, NSW Australia.
Fruit: Unripe fruit color intensity light Unripe fruit color 144A Cluster density is medium. Diameter is medium to large (mean 16.2 mm) Weight medium (approximately 1.9 g) Shape round Attitude of calyx erect Diameter of calyx basin medium (mean 5.5 mm) Depth of calyx basin is deep (to 2.2 mm) Intensity of bloom strong Intensity of blue color of skin with bloom removed is very dark Color of skin 202A Sweetness when ripe medium to high Firmness when ripe firm Acidity when ripe medium Size of scar is small (1.0-1.5 mm) Storage quality good.
   Date of 50% maturity.—approximately 25^{th} August at Lindendale, NSW Australia.
   Fruit development period.—approximately 55 days in protected cultivation at Lindendale, NSW Australia.
Seed: Seed color greyed orange 165B Seed abundance in fruit medium Seed size 1.6-1.8 mm long for fully developed seeds.

TABLE 1
Comparison with other varieties (extract from Australian PBR data)

| Organ/Plant Part: Context | Ridley 1104 | C99-42 | Star |
|---|---|---|---|
| Plant: vigor | strong | medium | medium |
| Plant: growth habit | spreading | semi upright | upright |
| Leaf: length | medium to long | medium | medium |
| Leaf: width | broad | narrow to | narrow |
| | | medium | |
| Leaf: shape | elliptic | elliptic | elliptic |
| Leaf: color of upper side | green | green | green |
| Leaf: intensity of green color on upper side | medium | medium | medium |
| (varieties with green leaf colour only) | | | |
| Leaf: margin | entire | entire | entire |
| Flower: size of corolla tube | medium to large | medium | medium |
| Flower: anthocyanin coloration of corolla tube | absent or very | absent or very | absent or very |
| | weak | weak | weak |
| Flower: ridges on corolla tube | present | present | present |
| Fruit cluster: density | medium | dense | medium |
| Flower: protusion of stigma | absent | absent | present |
| Unripe fruit: intensity of green colour | light | light | light |
| Fruit: size | medium to large | medium to large | large |
| Fruit: shape in longitudinal section | round | round | round |
| Fruit: attitude of sepals | erect | erect | erect |
| Fruit: diameter of calyx basin | small to medium | medium | large to very |
| | | | large |
| Fruit: depth of calyx basin | deep | medium | shallow |
| Fruit: intensity of bloom | strong | strong | strong |
| Fruit: color of skin | dark blue | dark blue | dark blue |
| Fruit: firmness | firm | firm | firm |
| Fruit: sweetness | medium to high | low to medium | low to medium |
| Fruit: acidity | medium | medium | medium |
| Fruit: size of scar | small | small | |
| Fruit: average weight of ripe berry (g) | 1.9 | 1.6 | 2.3 |
| Time of: beginning of flowering on current | early to medium | very early to early | early to medium |
| year's shoot (varieties which fruit on one- | | | |
| year-old and current season's shoots only) | | | |
| Time of: beginning of fruit ripening on | early to medium | early | early to medium |
| current year's shoot (varieties which fruit on | | | |
| one-year-old and current season's shoots) | | | |

What is claimed is:

1. A new and distinct cultivar of Vaccinium hybrid plant named ‘RIDLEY 1104’, substantially as herein shown and described.
